(19) United States
(12) Patent Application Publication
(10) Pub. No.: US 2004/0235957 A1
(43) Pub. Date: Nov. 25, 2004

(54) Use of sulfonamide derivatives as pharmaceuticals compounds

(76) Inventors: David Bleakman, Zionsville, IN (US); Amy Suzon Chappell, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/491,030

(22) PCT Filed: Sep. 30, 2002
(86) PCT No.: PCT/US02/28148

Related U.S. Application Data
(60) Provisional application No. 60/329,056, filed on Oct. 12, 2001.

(51) Int. Cl. A61K 31/18

(52) U.S. Cl. 514/602; 514/603

(57) ABSTRACT

The present invention provides a method of treating Bipolar I Disorder, Single Manic Episode; Bipolar I Disorder, Most Recent Episode Hypomanic; Bipolar I Disorder, Most Recent Episode Manic; Bipolar I Disorder, Most Recent Episode Mixed; Bipolar I Disorder, Most Recent Episode Depressed; Bipolar I Disorder, Most Recent Episode Unspecified; Bipolar II Disorder; or Bipolar Disorder NOS, in a patient, comprising administering to said patient an effective amount of a compound selected from the group consisting of: A) 2-[4-(4-{2-[(methylsulfonyl)amino]ethyl}phenyl)phenyl]propyl}[(methylethyl)sulfonyl]amine; B) {(2R)-2-[4-(4-{2-[(methylsulfonyl)amino]ethyl}phenyl)phenyl]propyl}[(methylethyl)sulfonyl]amine; D) N-2-(4-N-(3,5-Difluorobenzamido)phenyl)propyl-2-propanesulfonamide; and E) N-[4-((1R)-1-methyl-2-{[(methylethyl)sulfonyl]amino}ethyl)phenyl](3,5-difluorophenyl)carboxamide; and the pharmaceutically acceptable salts thereof.

The present invention relates to the use of certain sulfonamide derivatives and their active metabolites as pharmaceutical compounds.

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathway in mammals, and is referred to as an excitatory amino acid (EM). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic”. This type of receptor has been subdivided into at least three subtypes, which are defined by the depolarizing actions of the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

AMPA receptors are assembled from four protein sub-units known as GluR1 to GluR4, while kainic acid receptors are assembled from the sub-units GluR5 to GluR7, and KA-1 and KA-2. Wong and Mayer, Molecular Pharmacology 44: 505-510, 1993. It is not yet known how these sub-units are combined in the natural state. However, the structures of certain human variants of each sub-unit have been elucidated, and cell lines expressing individual sub-unit variants have been cloned and incorporated into test systems designed to identify compounds which bind to or interact with them, and hence which may modulate their function. Thus, European patent application, publication number EP-A2-0574257 discloses the human sub-unit variants GluR1B, GluR2B, GluR3A and GluR3B. European patent application, publication number EP-A1-0583917 discloses the human sub-unit variant GluR4B.

One distinctive property of AMPA and kainic acid receptors is their rapid deactivation and desensitization to glutamate. Yamada and Tang, The Journal of Neuroscience, September 1993, 13(9): 3904-3915 and Kathryn M. Partin, J. Neuroscience, Nov. 1, 1996, 16(21): 6634-6647. The physiological implications of rapid desensitization, and deactivation if any, are not fully understood.

It is known that the rapid desensitization and deactivation of AMPA and/or kainic acid receptors to glutamate may be inhibited using certain compounds. This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

AMPA receptor potentiators have been shown to improve memory in a variety of animal tests. Staubli et al., Proc. Natl. Acad. Sci., Vol. 91, pp 777-781, 1994, Neurobiology, and Arai et al., The Journal of Pharmacology and Experimental Therapeutics, 278: 627-638, 1996.

In addition, certain sulfonamide derivatives which potentiate glutamate receptor function in a mammal have been disclosed in the following International Patent Application Publications: WO 98/33496 published Aug. 6, 1998; WO 99/43285 published Sep. 2, 1999; WO 00/06539; WO 00/06537, WO 00/06176, WO 00/06159, WO 00/06158, WO 00/06157, WO 00/06156, WO 00/06149, WO 00/06148, and WO 00/06083, all published Feb. 10, 2000; WO 00/66546 published Nov. 9, 2000, and WO 01/42203 published Jun. 14, 2001.

The present invention provides a method of treating Bipolar I Disorder, Single Manic Episode; Bipolar I Disorder, Most Recent Episode Hypomanic; Bipolar I Disorder, Most Recent Episode Manic; Bipolar I Disorder, Most Recent Episode Mixed; Bipolar I Disorder, Most Recent Episode Depressed; Bipolar I Disorder, Most Recent Episode Unspecified; Bipolar II Disorder; or Bipolar Disorder NOS, in a patient, comprising administering to said patient an effective amount of a compound selected from the group consisting of:

A) 2-[4-(4-{2-[(methylsulfonyl)amino]ethyl}phenyl)phenyl]propyl}[(methylethyl)sulfonyl]amine;

B) {(2R)-2-[4-(4-{2-[(methylsulfonyl)amino]ethyl}phenyl)phenyl]propyl}[(methylethyl)sulfonyl]amine;

C) {(2S)-2-[4-(4-{2-[(methylsulfonyl)amino]ethyl}phenyl)phenyl]propyl}[(methylethyl)sulfonyl]amine;

D) N-2-(4-N-(3,5-Difluorobenzamido)phenyl)propyl-2-propanesulfonamide;

E) N-[4-((1R)-1-methyl-2-{[(methylethyl)sulfonyl]amino}ethyl)phenyl](3,5-difluorophenyl)carboxamide; and

F) N-[4-((1S)-1-methyl-2-{[(methylethyl)sulfonyl]amino}ethyl)phenyl](3,5-difluorophenyl)carboxamide; and

the pharmaceutically acceptable salts thereof.

The present invention further provides the use of compound A, B, C, D, E, or F as defined herein, or a pharmaceutically acceptable salt thereof for the manufacture of a medicament for treating Bipolar I Disorder, Single Manic Episode; Bipolar I Disorder, Most Recent Episode Hypomanic; Bipolar I Disorder, Most Recent Episode Manic; Bipolar I Disorder, Most Recent Episode Mixed; Bipolar I Disorder, Most Recent Episode Depressed; Bipolar I Disorder, Most Recent Episode Unspecified; Bipolar II Disorder; or Bipolar Disorder NOS.

In addition, the present invention provides the use of compound A, B, C, D, E, or F as defined herein, or a pharmaceutically acceptable salt thereof for treating Bipolar I Disorder, Single Manic Episode; Bipolar I Disorder, Most Recent Episode Hypomanic; Bipolar I Disorder, Most Recent Episode Manic; Bipolar I Disorder, Most Recent Episode Mixed; Bipolar I Disorder, Most Recent Episode Depressed; Bipolar I Disorder, Most Recent Episode Unspecified; Bipolar II Disorder; or Bipolar Disorder NOS.

The present invention also provides a method of treating multiple sclerosis, in a patient, comprising administering to said patient an effective amount of a compound selected from the group consisting of:

A) 2-[4-(4-{2-[methylsulfonyl)amino]ethyl}phenyl)phenyl]propyl}[(methylethyl)sulfonyl]amine;

B) {(2R)-2-[4-(4-{2-[methylsulfonyl)amino]ethyl}phenyl)phenyl]propyl}[(methylethyl)sulfonyl]amine;

C) {(2S)-2-[4-(4-{2-[methylsulfonyl)amino]ethyl}phenyl)phenyl]propyl}[(methylethyl)sulfonyl]amine;

D) N-2-(4-N-(3,5-Difluorobenzamido)phenyl)propyl-2-propanesulfonamide;

E) N-[4-((1R)-1-methyl-2-{[(methylethyl)sulfonyl]amino}ethyl)phenyl](3,5-difluorophenyl)carboxamide; and

F) N-[4-((1S)-1-methyl-2-{[(methylethyl)sulfonyl]amino}ethyl)phenyl](3,5-difluorophenyl)carboxamide; and

The pharmaceutically acceptable salts thereof.

The present invention further provides the use of compound A, B, C, D, E, or F as defined herein, or a pharmaceutically acceptable salt thereof for the manufacture of a medicament for treating multiple sclerosis.

As used herein the term “compound A” refers to 2-[4-(4-{2-[(methylsulfonyl)amino]ethyl}phenyl)phenyl]propyl}[(methylethyl)sulfonyl]amine.

As used herein the term “compound B” refers to {(2R)-2-[4-(4-{2-[(methylsulfonyl)amino]ethyl}phenyl)phenyl]propyl}[(methylethyl)sulfonyl]amine.

As used herein the term “compound C” refers to {(2S)-2-[4-(4-{2-[(methylsulfonyl)amino]ethyl}phenyl)phenyl]propyl}[(methylethyl)sulfonyl]amine.

As used herein the term “compound D” refers to N-2-(4-N-(3,5-Difluorobenzamido)phenyl)propyl-2-propanesulfonamide.

As used herein the term “compound E” refers to N-[4-((1R)-1-methyl-2-{[(methylethyl)sulfonyl]amino}ethyl)phenyl](3,5-difluorophenyl)carboxamide.

As used herein the term “compound F” refers to N-[4-((1S)-1-methyl-2-{[(methylethyl)sulfonyl]amino}ethyl)phenyl](3,5-difluorophenyl)carboxamide.

As used herein the term “compound A, B, C, D, E, or F” refers to the above-defined compounds A, B, C, D, E, or F.

As used herein, the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

As used herein the term “AMPA receptor potentiator” refers to a compound which inhibits the rapid desensitization or deactivation of AMPA receptors to glutamate.

As used herein the term “active metabolite” refers to a compound which results from the metabolism by a patient of compound A, B, C, D, E, or F after said compound is administered to the patient, and in addition, the resulting metabolite is useful in treating the targeted condition or disorder. For example, a variety of conditions may be treated by the active metabolite, such as those associated with glutamate hypofunction, such as psychiatric and neurological disorders, as discussed in further detail hereinbelow. It is understood that more than one active metabolite may result from the metabolism of each compound A, B, C, D, E, or F, and as such, multiple metabolites from a single compound are understood to be included within the term “active metabolite”.

A wide variety of conditions may be treated or prevented by compounds A, B, C, D, E, or F, or their corresponding active metabolites, or their pharmaceutically acceptable salts through their action as potentiators of glutamate receptor function. Such conditions include those associated with glutamate hypofunction, such as psychiatric and neurological disorders, for example cognitive disorders and neuro-degenerative disorders such as Alzheimer's disease; age-related dementias; age-induced memory impairment; cognitive deficits due to autism, mild cognitive impairment, Down's syndrome and other central nervous system disorders with childhood onset, cognitive deficits post electroconvulsive therapy, movement disorders such as tardive dyskinesia,

Huntington's chorea, myoclonus, dystonia, spasticity, andParkinson's disease; reversal of drug-induced states (such as cocaine, amphetamines, alcohol-induced states); depression; attention deficit disorder; attention deficit hyperactivity disorder; psychosis; cognitive deficits associated with psychosis; which includes, for example, cognitive impairment associated with schizophrenia; drug-induced psychosis; obesity; multiple sclerosis; stroke; cognitive deficits associated with stroke, traumatic spinal cord injury; traumatic brain injury; and sexual dysfunction. Compounds A, B, C, D, E, or F may also be useful for improving memory (both short term and long term) and learning ability, for example, as cognition enhancers. The present invention provides the use of compounds of compounds A, B, C, D, E, or F for the treatment of each of these conditions.

Additional conditions which may be treated by compounds A, B, C, D, E, or F, or their corresponding active metabolite, or their pharmaceutically acceptable salts include the following as listed in Table I. The conditions listed in Table I have been characterized in accordance with the Diagnostic and Statistical Manual of Mental Disorders, Fourth Edition, 1994, Washington, D.C., American Psychiatric Association, (referred to hereinafter as the DSM-IV). It is appreciated by one of ordinary skill in the art that certain conditions set forth in Table I fall within the scope of or overlap with the conditions listed hereinabove which may be treated by compounds A, B, C, D, E, or F, or their corresponding active metabolite. It is further appreciated by one of ordinary skill in the art that there are alternative nomenclatures, nosologies, and classification systems for pathologic psychological conditions and that these systems evolve with medical scientific progress.

TABLE I

| Diagnostic | |
|---|---|
| Code | Condition |
| 317 | Mild Mental Retardation |
| 318.0 | Moderate Mental Retardation |
| 319 | Mental Retardation, Severity Unspecified |
| 315.00 | Reading Disorder |
| 315.1 | Mathematics Disorder |
| 315.2 | Disorder of Written Expression |
| 315.9 | Learning Disorder NOS |
| 315.4 | Developmental Coordination Disorder |
| 315.31 | Expressive Language Disorder |
| 315.32 | Mixed Receptive-Expressive Language Disorder |
| 315.39 | Phonological Disorder |
| 307.0 | Stuttering |
| 307.9 | Communication Disorder NOS |
| 299.00 | Autistic Disorder |
| 299.80 | Rett's Disorder |
| 299.10 | Childhood Disintegrative Disorder |
| 299.80 | Asperger's Disorder |
| 299.80 | Pervasive Developmental Disorder NOS |
| 314.01 | Attention-Deficit/Hyperactivity Disorder, Combined Type |
| 314.00 | Attention-Deficit/Hyperactivity Disorder, Predominantly |
| | Inattentive Type |
| 314.01 | Attention-Deficit/Hyperactivity Disorder, Predominantly |
| | Hyperactive-Impulsive Type |
| 314.9 | Attention-Deficit/Hyperactivity Disorder NOS |
| 307.23 | Tourette's Disorder |
| 307.22 | Chronic Motor or Vocal Tic Disorder |
| 307.21 | Transient Tic Disorder |
| 307.20 | Tic Disorder |
| 290.10 | Dementia of the Alzheimer's Type, With Early Onset, |
| | Uncomplicated |
| 290.11 | Dementia of the Alzheimer's Type, With Early Onset, With |
| | Delirium |
| 290.0 | Dementia of the Alzheimer's Type, With Late Onset, |
| | Uncomplicated |
| 290.3 | Dementia of the Alzheimer's Type, With Late Onset, With |
| | Delirium |
| 290.20 | Dementia of the Alzheimer's Type, With Late Onset, With |
| | Delusions |
| 290.21 | Dementia of the Alzheimer's Type, With Late Onset, With |
| | Depressed Mood |
| 290.40 | Vascular Dementia, Uncomplicated |
| 290.41 | Vascular Dementia, With Delirium |
| 290.42 | Vascular Dementia, With Delusions |
| 290.43 | Vascular Dementia, With Depressed Mood |
| 294.1 | Dementia Due to HIV Disease |
| 294.1 | Dementia Due to Head Trauma |
| 294.1 | Dementia Due to Parkinson's Disease |
| 294.1 | Dementia Due to Huntington's Disease |
| 290.10 | Dementia Due to Pick's Disease |
| 290.10 | Dementia Due to Creutzfeldt-Jakob Disease |
| 294.1 | Dementia due to other General Medical Conditions |
| 294.8 | Dementia NOS |
| 294.0 | Amnestic Disorder Due to a General Medical Condition |
| 294.8 | Amnestic Disorder NOS |
| 304.10 | Sedative, Hypnotic, or Anxiolytic Dependence |
| 295.30 | Schizophrenia, Paranoid Type |
| 295.10 | Schizophrenia, Disorganized Type |
| 295.20 | Schizophrenia, Catatonic Type |
| 295.90 | Schizophrenia, Undifferentiated Type |
| 295.60 | Schizophrenia, Residual Type |
| 295.40 | Schizophreniform Disorder |
| 295.70 | Schizoaffective Disorder |
| 297.1 | Delusional Disorder |
| 298.8 | Brief Psychotic Disorder |
| 297.3 | Shared Psychotic Disorder |
| 293.81 | Psychotic Disorder, With Delusions |
| 293.82 | Psychotic Disorder, With Hallucinations |
| 298.9 | Psychotic Disorder NOS |
| 296.2x | Major Depressiv Disorder, Single Episode |
| 296.3x | Major Depressive Disorder, Recurrent |
| 300.4 | Dysthymic Disorder |
| 311 | Depressive Disorder NOS |
| 296.0x | Bipolar I Disorder, Single Manic Episode |
| 296.40 | Bipolar I Disorder, Most Recent Episode Hypomanic |
| 296.4x | Bipolar I Disorder, Most Recent Episode Manic |
| 296.6x | Bipolar I Disorder, Most Recent Episode Mixed |
| 296.5x | Bipolar I Disorder, Most Recent Episode Depressed |
| 296.7 | Bipolar I Disorder, Most Recent Episode Unspecified |
| 296.89 | Bipolar II Disorder |
| 301.13 | Cyclothymic Disorder |
| 296.80 | Bipolar Disorder NOS |
| 293.83 | Mood Disorder Due to a General Medical Condition |
| 296.90 | Mood Disorder NOS |
| 307.44 | Primary Hypersomnia |
| 347 | Narcolepsy |
| V62.89 | Borderline Intellectual Functioning |
| 780.9 | Age-Related Cognitive Decline |
| V62.3 | Academic Problem |

The present invention includes the pharmaceutically acceptable salts of the compounds A, B, C, D, E, and F, and the corresponding active metabolite. A compound of this invention can possess a sufficiently acidic group, a sufficiently basic group, or both functional groups, and accordingly react with any of a number of organic and inorganic bases, and inorganic and organic acids, to form a pharmaceutically acceptable salt. The term “pharmaceutically acceptable salt” as used herein, refers to salts of the compounds of the above formula which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts. Such salts include the pharmaceutically acceptable salts listed in Journal of Pharmaceutical Science, 66, 2-19 (1977), which are known to the skilled artisan. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, oacetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, a-hydroxybutyrate, glycolate, tartrate, benzenesulfonate, methanesulfonate, ethanesulfonate, propanesulfonate, hydroxyethanesulfonate, 1-naphthalenesulfonate, 2-napththalenesulfonate, 1,5-naphthalenedisulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form. As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

Compounds A, B, C, D, E, and F, or the corresponding active metabolite, or their pharmaceutically acceptable salts can be prepared by one of ordinary skill in the art following art recognized techniques and procedures. Such techniques and procedures can be found for example in International Patent Application Publications: WO 98/33496 published Aug. 6, 1998; WO 99/43285 published Sep. 2, 1999; WO 00/06539; WO 00/06537, WO 00/06176, WO 00/06159, WO 00/06158, WO 00/06157, WO 00/06156, WO 00/06149, WO 00/06148, and WO 00/06083, all published Feb. 10, 2000; and WO 00/66546 published Nov. 9, 2000. The reagents and starting materials are readily available to one of ordinary skill in the art.

In addition, active metabolites of compounds A, B, C, D, E, and F can be isolated and purified from the blood, serum or urine of patients to whom compounds A, B, C, D, E, or F have been administered. Isolation and purification is readily accomplished by one of ordinary skill in the art using techniques and procedures well known in the art, such as extraction techniques and chromatography, such as high performance liquid chromatography or flash chromatography to provide the purified active metabolites of compounds A, B, C, D, E, and F. The pharmaceutically acceptable salts are then readily prepared by one of ordinary skill in the art using well known techniques and procedures.

The following examples further illustrate the invention and represent typical syntheses of compounds A, B, C, D, E, and F. The reagents and starting materials are readily available to one of ordinary skill in the art. As used herein the term “Chromatotron®” (Harrison Research Inc., 840 Moana Court, Palo Alto Calif. 94306) is recognized by one of ordinary skill in the art as an instrument which is used to perform centrifugal thin-layer chromatography. As used herein, the following terms have the meanings indicated: “eq” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “aq” refers to aqueous; “iPrOAc” refers to isopropyl acetate; “MTBE” refers to tert-butyl methyl ether; “methyl DAST” refers to dimethylaminosulfur trifluoride, “DAST” refers to diethylaminosulfur trifluoride, “DBU” refers to 1,8-diazabicyclo[5.4.0]undec-7-ene; as used herein “Pd(dppf)₂Cl₂ catalyst” refers to ([1,1′-bis(diphenylphosphino)ferrocene]dichloropalladium(II) complex with CH₂Cl₂; as used herein the terms “Me”, “Et”, “Pr”, “iPr”, and “Bu” refer to methyl, ethyl, propyl, isopropyl, and butyl respectively, and “RT” refers to room temperature.

### EXAMPLE 1

### Preparation of N-2-(4-N-(3,5-Difluorobenzamido)phenyl)propyl-2-propanesulfonamide

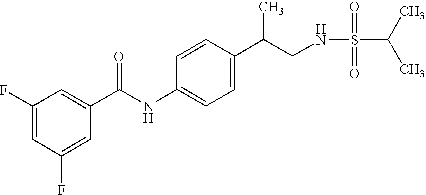

The title compound is prepared in a manner analogous to the procedure described at Example 196 in International Patent Application Publication WO 98/33496 published Aug. 6, 1998 from 3,5-difluorobenzoyl chloride.

Alternatively, the title compound can be prepared as described in examples 2 and 3 below without employing the resolution steps as would be appreciated by one of ordinary skill in the art.

More specifically, into a 500 mL 3-neck flask fitted with a stirrer and thermometer, 3,5-difluorobenzoyl chloride (1.13 g) was added dropwise to a stirred solution of [2-(4-aminophenyl)propyl][(methylethyl)sulfonyl]amine (1.50 g) and triethylamine (625 mg) in methylene chloride (200 mL) at room temperature and under a nitrogen atmosphere. After stirring one hour at this temperature, TLC showed that the starting aniline had been consumed. The organic layer was washed once with water, dried over potassium carbonate, and concentrated under reduced vacuum to yield the crude material (2.61 g) as a solid. This crude material was purified by recrystallization from hexanelethyl acetate 1:1 to yield the title compound (1.64 g, 71%)) as yellow crystals. M.P. 158° C.-160° C.

Ion spray M.S. 397.1 (M*+1).

Calculated for C₁₉H₂₂N₂O₂SF₂-H₂O: Theory: C 55.03, H 5.83, N 6.76. Found: C 54.63, H 5.84, N 6.61.

### EXAMPLE 2

### Preparation of N-[4-((1R)-1-methyl-2-{[(methylethylsulfonyl]amino}ethyl)phenyl](3,5-difluorophenyl)carboxamide

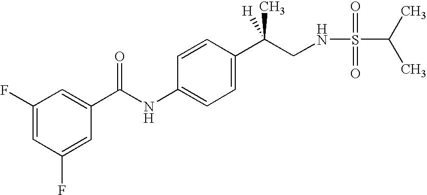

### Preparation of 2-Phenyl-1-propylamine HCl

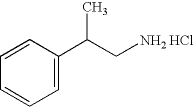

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitated solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder. ¹H NMR analysis of the free base: ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

### Preparation of (2R)-2-phenylpropylamine malate

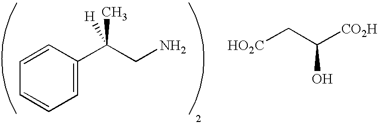

Scheme I, step B: To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison, via chiral HPLC, with commercially available (R)-2-phenyl-1-propylamine).

¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (˜79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10 H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

### Preparation of ((2R)-2-phenylpropyl)[(methylethyl)sulfonyl]amine

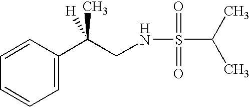

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO4), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

### Preparation of [(2R)-2-(4-aminophenyl)propyl][(methylethyl)sulfonyl]amine p-toluenesulfonate

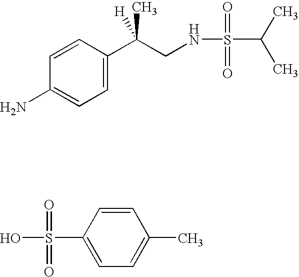

To a round-bottom flask equipped with stir rod, thermocouple and nitrogen purge at 25° C., was charged ((2R)-2-phenylpropyl)[(methylethyl)sulfonyl]amine (5.00 g, 0.0207 mol), trifluoroacetic acid (15 mL), dichloromethane (1.2 mL) and heptane (8 mL). The mixture was cooled to −5° C. and 98% fuming nitric acid (1.60 g, 0.0249 mol) was added dropwise. The reaction mixture was stirred at −5 to +5° C. for 3-5 hours and then warmed to 20-25° C. The reaction was allowed to stir until GC analysis revealed that ((2R)-2-phenylpropyl)[(methylethyl)sulfonyl]amine is less then 1% (area %).

The reaction mixture was then diluted with dichloromethane (20 mL) and diionized water (20 mL), and the mixture was transferred to a suitably sized 3-neck bottom outlet round-bottom flask. The mixture was stirred for 10-15 minutes. The aqueous phase was separated, extracted with dichloromethane (1×20 mL), and the organic phases were combined. To the organic phase was added water (15 mL), 10% NaOH (10 mL), and the pH was adjusted to 6.5-7.5 with saturated sodium carbonate. After 10-15 minintes of stirring, the organic layer was separated and concentrated to an oil under reduced pressure (25-35° C.).

The oil containing the mixture of [(2R)-2-(4-nitrophenyl)propyl][(methylethyl)sulfonyl]amine, [(2R)-2-(3-nitrophenyl)propyl][(methylethyl)sulfonyl]amine, and [(2R)-2-(2-nitrophenyl)propyl][(methylethyl)sulfonyl]amine, was diluted with ethanol and was transferred to a Parr bottle containing 1.25 g of 5% Pd on C (rinsed in with 5 mL of THF) under nitrogen (total ethanol=45 mL). The reaction mixture was hydrogenated for 16-20 hours at 20-25° C. until the GC area % of [(2R)-2-(4-aminophenyl)propyl][(methylethyl)sulfonyl]amine was greater than 70%. The reaction mixture was filtered through Hyflo followed by an ethanol rinse (25 mL).

The oil was diluted with THF (35 mL) and p-toluenesulfonic acid monohydrate (3.94 g, 0.0207 mol) was added with stirring at 20-25° C. When the solids completely dissolved, MTBE (22 mL) was added and the slurry was stirred for 1-2 hours. The slurry was filtered and the cake was rinsed three times with a 3:7 (v/v) solution of MBTE and THF. This process afforded [(2R)-2-(4-aminophenyl)propyl][(methylethyl)sulfonyl]amine p-toluenesulfonate in 53.5% yields as an off white powder. Chiral analysis of the freebase, [(2R)-2-(4-aminophenyl)propyl][(methylethyl)sulfonyl]amine, obtained extractively from [(2R)-2-(4-aminophenyl)propyl][(methylethyl)sulfonyl]amine p-toluenesulfonate, showed % e.e. of 99.5%.

¹H NMR (CD₃OD, 300 MHz) δ 7.70 (d, 2H, J=8.4), 7.43 (d, 2H, J=8.4), 7.33 (d, 2H, J=8.4), 7.23 (d, 2H, J=7.8), 3.22 (m, 2H), 3.08 (quint, 1H, J=6.9), 2.99 (q, 1H, J=6.9), 1.29 (d, 3H, J=6.6), 1.23 (d, 3H, J=6.6).

### Preparation of [(2R)-2-(4-aminophenyl)propyl][(methylethyl)sulfonyl]amine

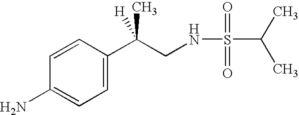

To a suspension of [(2R)-2-(4-aminophenyl)propyl][(methylethyl)sulfonyl]amine p-toluenesulfonate (41.2 g, 0.0961 mol) in CH₂Cl₂ (300 mL) was added saturated aqueous NaHCO₃ until the pH of the aqueous phase was 6.5. The phases were separated and the organic phase was washed with 5% NaHCO₃ (2×100 mL), H₂O (100 mL), and concentrated to provide [(2R)-2-(4-aminophenyl)propyl][(methylethyl)sulfonyl]amine as an oil. After diluting the oil with diethyl ether (50 mL), crystallization began after 10 min. Caution: Heat of crystallization caused ether to boil. After the exotherm subsided (45 minutes), the suspension was filtered, and the filter cake was washed with diethyl ether (2×20 mL), and dried under reduced pressure to afford [(2R)-2-(4-aminophenyl)propyl][(methylethyl)sulfonyl]amine (21.7 g, 88.1%). ¹H NMR (CDCl₃, 300 MHz) δ 7.00 (d, 2H, J=8.1); 6.66 (d, 2H, J=8.4), 3.83 (m, 1H), 3.65 (br s, 2H), 3.31 (m, 1H), 3.09 (m, 2H), 2.85 (m, 1H), 1.30 (d, 3H, J=7.2), 1.26 (d, 3H, J=6.9), 1.24 (d, 3H, J=6.9).

### Preparation of N-[4-((1R)-1-methyl-2-{[(methylethyl)sulfonyl]amino}ethyl)phenyl](3,5-difluorophenyl)carboxamide, Method A

[(2R)-2-(4-aminophenyl)propyl][(methylethyl)sulfonyl]amine p-toluenesulfonate (60.0 g, 0.140 mol), suspended in dichloromethane (375 mL), was treated with saturated aqueous NaHCO₃ in an amount sufficient to bring the salt into solution. The organic phase was separated and washed twice with aqueous NaHCO₃. HPLC analysis showed complete removal of p-toluenesulfonate from the organic phase. The organic phase was dried (MgSO₄), filtered, and chilled to −10° C. 3,5-difluorobenzoyl chloride (27.2 g, 0.154 mol) was added dropwise over 10 min and the mixture was allowed to warm to room temperature with stirring overnight.

After completion of reaction, the mixture was diluted with water (100 mL) and acetone (75 mL). The phases were separated, and the organic phase was washed with 0.1N HCl (2×100 mL), 0.01N NaOH (3×100 mL), and 0.1 N HCl (1×100 mL). The organic phase was separated and concentrated to a solid. The solid was resuspended in ethyl acetate and co-evaporated twice with ethyl acetate (2×60 mL) to remove traces of dichloromethane. The residue was transferred to a 500 mL flask with ethyl acetate (150 mL) and this mixture was heated to reflux to afford a clear solution. The solution was allowed to cool to room temperature over 5 hours, and the suspension was left to stir slowly overnight. The suspension was cooled to 0° C. and stirred for 1 hour. The product was collected by filtration and was vacuum dried to afford N-[4-((1R)-1-methyl-2-{[(methylethyl)sulfonyl]amino}ethyl)phenyl](3,5-difluorophenyl)carboxamide (43.9 g, 79.0%) as a white crystalline solid.

¹H NMR (CDCl₃, 300 MHz) δ 7.80 (s, 1H), 7.59 (d, 2H, J=8.4), 7.40 (m, 2H), 7.23 (d, 2H, J=8.7), 7.01 (tt, 1H, J=2.1, 8.7), 3.87 (dd, 1H, J=5.1, 7.5), 3.36 (m, 1H), 3.21 (m, 1H), 3.09 (m, 1H), 2.98 (m, 1H), 1.32 (d, 3H, J=6.6), 1.30 (d, 3H, J=7.2), 1.28 (d, 3H, J=6.6).

### Preparation of N-[4-((1R)-1-methyl-2-{[(methylethyl)sulfonyl]amino}ethyl)phenyl](3,5-difluorophenyl)carboxamide, Method B

To a 0° C. solution of [(2R)-2-(4-aminophenyl)propyl][(methylethyl)sulfonyl]amine (21.5 g, 0.0838 mol) and triethylamine (9.75 g, 13.4 mL, 0.0964 mol) in CH₂Cl₂ (86 mL) was added 3,5-difluorobenzoyl chloride (16.3 g, 0.0922 mol) dropwise over 30 min. After the addition was complete, the reaction mixture was stirred at 20° C. for 1 hour. The reaction mixture was washed with deionized water (2×100 mL) and 0.1 N HCl (2×100 mL). The organic phase was diluted with acetone (50 mL) to ensure complete dissolution of the product and the organic phase was washed with saturated K₂CO₃ (100 mL), 0.1 N HCl (100 mL), dried (MgSO₄, 3 g), filtered and co-evaporated with EtOAc to afford an oil. This oil was diluted with diethyl ether (125 mL), which induced crystallization. The solids were collected by filtration, washed with diethyl ether (2×20 mL), and dried under reduced pressure at room temperature overnight to afford N-[4-((1R)-1-methyl-2-{[(methylethyl)sulfonyl]amino}ethyl)phenyl](3,5-difluorophenyl)carboxamide (31.8 g, 95.7%) as a white crystalline powder.

An analytical sample was prepared via recrystallization from EtOAc. Thus, a clear solution of N-[4-((1R)-1-methyl-2-{[(methylethyl)sulfonyl]amino}ethyl)phenyl](3,5-difluorophenyl)carboxamide (28 g) was achieved in refluxing EtOAc (90 mL, minimum amount). This solution was allowed to cool over 2 hour to room temperature without stirring. The resulting dense mass was pulvarized with a glass rod and recovered by filtration. The collected solids were reslurried in diethyl ether, filtered and dried under reduced pressure to afford N-[4-((1R)-1-methyl-2-{[(methylethyl)sulfonyl]amino}ethyl)phenyl](3,5-difluorophenyl)carboxamide (22.2 g, 79% recovery) as a white crystalline powder.

In addition, the final title compound, N-[4-((1R)-1-methyl-2-{[(methylethyl)sulfonyl]amino}ethyl)phenyl](3,5-difluorophenyl)carboxamide, can be jet milled by one of ordinary skill in the art, for example, with a Model 4 SDM Micronizer by Sturtevant Inc. to provide compound with a mean particle size of about 5.5 microns.

### EXAMPLE 3

### Alternative Preparation of N-[4-((1R)-1-methyl-2-{[(methylethyl)sulfonyl]amino}ethyl)phenyl](3,5-difluoroghenyl)carboxamide

### Preparation of (2R)-2-(4-nitrophenyl)propanoic acid, S(−)-α-methylbenzylamine

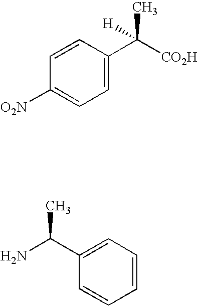

A 2 liter three necked flask equipped with a mechanical stirrer is charged with racemic 2-(4-nitrophenyl)propionic acid (40.55 grams, 0.208 mol) and ethyl acetate (1600.0 mL). To this solution at 30° C. was then added S(−)-α-methylbenzylamine (13.49 mL, 0.104 mol) all at once. Reaction exothermed to 38° C. with massive formation of a white precipitate in less than 15.0 minutes. The reaction mixture was then heated at ethyl acetate reflux for 10.0 minutes and allowed to equilibrate to room temperature with stirring overnight. The precipitate was then filtered to give a semi-dried white product, (2R)-2-(4-nitrophenyl)propanoic acid, S(−)-α-methylbenzylamine (wet cake=25.43 grams). Reslurried the wet cake in ethyl acetate (1600.0 mL) at reflux for 10.0 minutes, stirred to room temperature overnight, and filtered the white precipitate, (2R)-2-(4-nitrophenyl)propanoic acid, S(−)-α-methylbenzylamine, (wet cake=21.02 grams, ee=91.4%). Repeated the later again and dried the precipitate at 40° C. in a vacuum oven for 24.0 hours, (2R)-2-(4-nitrophenyl)propanoic acid, S(−)-α-methylbenzylamine, (18.02 g, 55%, ee=95%); ¹H nmr (DMSO, 300 MHz) δ 1.31-1.32 (d, 3H), 1.37-1.38 (d, 3H), 3.56-3.60 (m, 1H), 4.18-4.20 (m, 1H), 7.27-7.53 (aromatic, 7H), 8.09-8.12 (aromatic, 2H); 13C nmr (DMSO, 300 MHz)) δ 19.91, 22.93, 48.45, 50.55, 123.71, 124.15, 127.15, 128.27, 129.06, 129.41, 129.76, 146.31, 153.36, 176.24.

### Preparation of (2R)-2-(4-nitrophenyl)propanoic acid

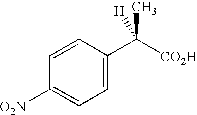

To reaction mixture of (2R)-2-(4-nitrophenyl)propanoic acid, S(−)-α-methylbenzylamine (56.04 g, 0.177 moles) in methylene chloride (400.0 mL) at room temperature was added 1 N HCl (300.0 mL) all at once with stirring for 45.0 minutes. The lower organic layer was then separated, dried with anhydrous magnesium sulfate, filtered, and concentrated under reduced pressure to afford the title compound, (2R)-2-(4-nitrophenyl)propanoic acid, (34.58 g, 100%) as an oil.

### Preparation of (2R)-2-(4-nitrophenyl)propan-1-ol

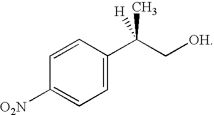

A 500 ml three necked round bottom flask equipped with a mechanical stirrer, thermometer, addition funnel, reflux condenser and a continuous nitrogen purge is charged with (2R)-2-(4-nitrophenyl)propanoic acid (8.12 g, 41.6 mmol) and THF (120.0 mL). To this solution was added 10.0M borane dimethylsulfide (10.56 ml, 105.66 mmol) over a period of 30.0 minutes at room temperature. Reaction is quite exothermic with evolution of gas (exotherm can be controlled by the rate of addition of borane solution). The reaction is then refluxed for 5.0 hours, brought to room temperature and then quenched very carefully with saturated potassium carbonate solution (100.0 mL). Foaming observed during the quench can be controlled by the rate of addition of the carbonate solution. After 3.0 hours of stirring, the top organic layer is separated and the aqueous layer back extracted with methylene chloride (130.0 mL). The combined organic layer is then washed with saturated brine (100.0 mL), dried over anhydrous magnesium sulfate, filtered, and concentrated under reduced pressure at 50° C. to afford (2R)-2-(4-nitrophenyl)propan-1-ol (7.24 g, 96%); ¹H nmr (CDCl₃, 300 MHz) δ 1.29 (d, 3H, J=7.02 Hz), 1.69 (b. triplet, OH), 3.05 (m, 1H), 3.72 (m, 2H), 7.39 (d, 2H), 8.15 (d, 2H); ¹³C nmr (CDCl₃, 300 MHz) δ 17.61, 25.82, 42.61, 68.17, 123.92, 128.61, 146.90, 152.24.

### Preraration of 2-[(2R)-2-(4-nitrophenyl)propyl]isoindoline-1,3-dione

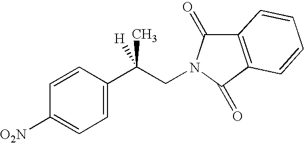

A 250 mL three necked round bottom flask equipped with a mechanical stirrer, addition funnel, thermometer, and a reflux condenser is charged with (2R)-2-(4-nitrophenyl)propan-1-ol (2.0 g, 11.04 mmol), phthalimide (1.62 g, 11.04 mm), triphenylphosphine (4.3 g, 16.59 mmol) and THF (50.0 mL) at room temperature. To this solution was added DEAD (2.6 mL, 16.59 mmol) over a period of 5 minutes (reaction exothermed to reflux by the end of addition). The reaction was then stirred to room temperature overnight for convenience, quenched with water (50.0 mL) and extracted organic with methylene chloride (50.0 mL). The organic layer was then dried with anhydrous magnesium sulfate, filtered, and concentrated under reduced pressure at 50° C. to an oil (11.62 g). Plug filtration of the oil over silica gel with 1:1 ethyl acetate/hexane (470.0 mL) and subsequent concentration of the fractions containing product afforded a light yellow precipitate. The precipitate was then dried in a house vacuum at 40° C. to provide 2-[(2R)-2-(4-nitrophenyl)propyl]isoindoline-1,3-dione (3.32 g, 96.9%); ¹H nmr (CDCl₃, 300 MHz) δ 1.50 (d, 3H, J=6.74Hz), 3.45 (m, 1H), 3.89-3.95 (m, 2H), 7.5 (d, 2H), 7.67 (m, 2H), 7.68 (m, 2H), 8.10 (d, 2H); ¹³C nmr (CDCl₃, 300 MHz) δ 19.21, 38.90, 44.45, 123.59, 123.99, 128.50, 131.86, 134.35, 151.13, 168.34.

### Preparation of (2R)-2-(4-nitrophenyl)propylamine

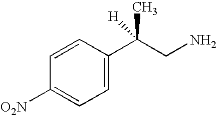

A 250 mL three necked round bottom flask equipped with a mechanical stirrer, thermometer, reflux condenser and addition funnel is charged with 2-[(2R)-2-(4-nitrophenyl)propyl]isoindoline-1,3-dione (25.02 g, 80.6 mmol) and toluene (200.0 mL). To this solution at room temperature was added anhydrous hydrazine (7.08 mL, 226.0 mmol). Reaction exothermed slightly and was stirred for 45 minutes, heated at 90° C.-95° C. until the disappearance of starting material. A massive precipitate formed by the end of the reaction. Cooled to room temperature and chilled to 0° C. before filtration. Concentration of the filtrate afforded (2R)-2-(4-nitrophenyl)propylamine (14.11 g, 97%) as an oil; ¹H nmr (CDCl₃, 300 MHz) δ 1.01 (b, 1H), 1.27 (d, 3H, J=6.4Hz), 2.87 (m, 2H), 7.36 (d, 2H), 8.14 (d, 2H); ¹³C nmr (CDCl₃, 300 MHz) δ 19.03, 43.51, 49.21, 123.67, 128.09, 153.04.

### Preparation of [(2R)-2-(4-nitrophenyl)propyl][(methylethyl)sulfonyl]amine

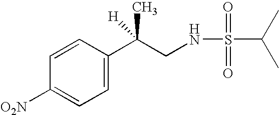

A 500 mL three necked round bottom flask equipped with a mechanical stirrer, thermometer and an addition funnel is charged with (2R)-2-(4-nitrophenyl)propylamine (11.75 g, 65.21 mmol), methylene chloride (150.0 mL) and triethylamine (18.2 mL, 130.4 mmol). To this solution at 0° C. was added isopropylsulfonyl chloride (8.92 mL, 63.9 mmol) over a period of 20 minutes. Reaction was then stirred to room temperature overnight, then quenched with 1N HCl (150.0 mL). The lower organic layer is separated and dried with anhydrous magnesium sulfate, filtered, and concentrated under reduced pressure to afford [(2R)-2-(4-nitrophenyl)propyl][(methylethyl)sulfonyl]amine (14.11 g, 97%) as an oil; ¹H nmr (CDCl₃, 300 MHz) δ 1.26 (d, 3H, J=6.7Hz), 1.31 (d, 6H), 3.06 (m, 1H), 3.30 (m, 1H), 4.25 (broad triplet, 1H), 7.38 (d, 2H), 8.10 (2H); ¹³C nmr (CDCl₃, 300 MHz) δ 16.75, 18.95, 41.29, 50.15, 53.85, 124.22, 128.52, 151.26.

### Preparation of [(2R)-2-(4-nitrophenyl)propyl][(methylethyl)sulfonyl]amine

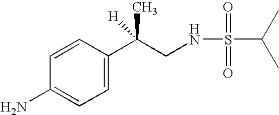

A 500 mL parr bottle is charged with [(2R)-2-(4-nitrophenyl)propyl][(methylethyl)sulfonyl]amine (14.45 g, 50.60 mmol), 3A EtOH (80.0 mL) and 10% P/C (4.0 9). The reaction mixture was then hydrogenated at room temperature and at 55 psi for 6 hours. Filtered reaction mixture over hyflo and washed cake with 3A EtOH (100.0 mL). The filtrate was then concentrated at reduced pressure to provide [(2R)-2-(4-nitrophenyl)propyl][(methylethyl)sulfonyl]amine (12.97 g, 100%) as an oil; ¹H nmr (CDCl₃, 300 MHz) δ 1.26 (d, 3H, J=6.7Hz), 1.31 (d, 6H), 2.4 (m, 1H), 3.0-3.2 (m, 2H), 3.2-3.4 (m, 1H), 4.0 (b, 1H), 4.6 (b, 2H), 6.61 (d, 2H), 7.0 (d, 2H).

### Preparation of N-[4-((1R)-1-methyl-2-{[(methylethyl)sulfonyl]amino}ethyl)phenyl](3,5-difluorophenyl)carboxamide

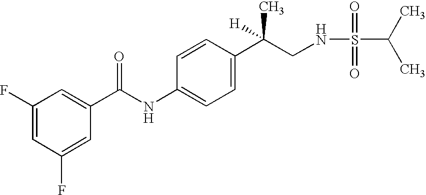

A 500 mL three necked round bottom flask equipped with a magnetic stirrer, thermometer, addition funnel and a positive nitrogen was charged with [(2R)-2-(4-nitrophenyl)propyl][(methylethyl)sulfonyl]amine (12.02 g, 46.85 mmol) and methylene chloride (200.0 mL). To this solution was added triethylamine (6.53 mL, 46.85 mmol) all at once. The solution was stirred for 10 minutes then added dropwise, neat 3,5-difluorobenzoyl chloride (5.9 mL, 46.85 mmol) over a period of 20 minutes. The reaction exothermed to reflux by the end of addition. Stirred to room temperature over the weekend for convenience. Quenched reaction with 1N HCl (100.0mL) and separated lower organic layer. Washed the organic layer with 25% brine (70.0 mL) and dried with anhydrous magnesium sulfate. Filtered precipitates and concentrated filtrate to a tan oil (20.0 g). To this oil was added 1:1 ethyl acetate/hexane (125.0 mL) with stirring. A massive off white precipitate formed. The precipitate was then filtered and the cake washed with 1:1 ethyl acetate/hexane (50.0 mL). Precipitate was then dried in a house vacuum oven at 40° C. to provide N-[4-((1R)-1-methyl-2-{[(methylethyl)sulfonyl]amino}ethyl)phenyl](3,5-difluorophenyl)carboxamide (15.02 g, 80.9%); ¹H NMR (CDCl₃, 300 MHz) δ 1.26-1.27 (d, 6H), 1.29-1.30 (d, 2H), 2.92 (m, 1H), 3.10 (m, 1H), 3.20 (1H), 3.3-3.4 (m, 1H), 7.0 (triplet, 1H), 7.20 (d, 2H), 7.40 (d, 2H), 7.60 (m, 2H), 8.19 (s, 1H); ¹³C NMR (CDCl₃, 300 MHz) δ 17.19, 17.30, 19.75, 41.03, 50.99, 54.15, 107.68, 107.86, 108.08, 111.12, 111.33, 121.81, 128.59, 136.97, 138.77, 140.51, 162.62, 162.71, 164.12, 164.61, 164.71.

### EXAMPLE 4

### Alternative Preparation of [(2R)-2-(4-aminophenyl)propyl][(methylethyl)sulfonyl]amine p-toluenesulfonate

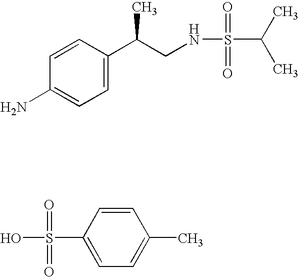

To a mechanically stirred solution of 2-phenyl-1-propylamine amine (50.0 g, 0.370 mol, can be prepared in a manner analogous to the procedure disclosed by A. W. Weston, et al., J. Am. Chem. Soc., 65, 674 (1943)) in 90% ethanol/H₂O (denatured with 0.5% toluene) (450 mL) was added L-malic acid (24.8 g, 0.185 mol) portionwise at room temperature with a 90% ethanol/H₂O rinse (50 mL) to give a clear solution after a mild exotherm. This solution was allowed to cool and a white precipitate appeared after 30 min. The precipitation was allowed to proceed with slow stirring overnight. The resulting slurry was suction filtered (buchner funnel) and rinsed with 100% ethanol (denatured with 0.5% toluene) (2×100 mL) to afford, after air-drying, 30 g of (2R)-2-phenylpropylamine malate as a white solid. Chiral chromatographic analysis of the isopropylsulfonamide derivative of the free base indicated 84% ee.

This (2R)-2-phenylpropylamine malate (30 g) was suspended in 90% ethanol/H₂O (300 mL) and heated to 78° C. with slow stirring to afford a clear colorless solution. The solution was allowed to cool slowly to room temperature overnight. Precipitation commenced at 60-65° C. IThe solids were filtered and rinsed at room temperature with 100% ethanol (2×50 mL) to give (2R)-2-phenylpropylamine malate (24.3 g, 32%) as a white crystalline solid. Chiral chromatographic analysis of the isopropylsulfonamide derivative of the free base indicated 96.5% ee.

### Preparation of (2R)-2-phenylpropylamine

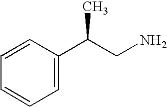

To a stirred suspension of (2R)-2-phenylpropylamine malate (24.3 g, 0.0601 mol, prepared directly above) in CH₂Cl₂ (200 mL) was added 1.0 N NaOH dropwise at room temperature. The organic phase was isolated, extracted with brine (1×125 mL), dried (Na₂SO₄), filtered, and concentrated under reduced pressure to give (2R)-2-phenylpropylamine (19 g) as a clear, colorless oil.

### Preparation of ((2R)-2-phenylpropyl)[(methylethyl)sulfonyl]amine

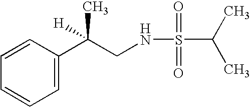

To a stirred 2° C. solution of (2R)-2-phenylpropylamine (0.12 mol) and triethylamine (24.3 g, 0.240 mol) in CH₂Cl₂ (140 mL) under nitrogen, was added a solution of isopropylsulfonyl chloride (97%) (16.3 g, 0.118 mol) in CH₂Cl₂ (20 mL) dropwise while maintaining the reaction temperature below 15° C. Residual isopropylsulfonyl chloride was rinsed in with CH₂Cl₂ (10 mL). This solution was stirred at 0° C. for 1 hour and was then allowed to warm to room temperature overnight.

The reaction mixture was re-cooled to 0° C. before adding 1 N HCl (125 mL) dropwise with stirring, The organic phase was then isolated and washed with saturated aqueous NaHCO₃ (1×125 mL) and the organic phase was separated, dried (MgSO₄), and filtered. The filtrate was concentrated under reduced pressure to afford ((2R)-2-phenylpropyl)[(methylethyl)sulfonyl]amine (25.76 g, 90%) as a yellow oil.

### Preparation of [(2R)-2-(4-aminophenyl)propyl][(methylethyl)sulfonyl]amine p-toluenesulfonate

To a room temperature solution of ((2R)-2-phenylpropyl)[(methylethyl)sulfonyl]amine (43.3 g, 0.179 mol) in trifluoroacetic acid (344 mL) was added NaNO₃ (45.7 g, 0.538 mol), and the resulting reaction mixture was stirred for 5 hours. The reaction mixture was diluted with CH₂Cl₂ (1 L), and washed with H₂O (2×300 mL), and separated. The organic phase was diluted again with H₂O (150 mL), and the heterogeneous mixture was neutralized with solid NaHCO₃ until the aqueous layer was pH 5.7. The organic phase was concentrated to an oil (43 g) that was dissolved in 3A ethanol (250 mL). The solution was then hydrogenated overnight at 50-60 psi over 7 g of 5% palladium on carbon.

¹H NMR analysis of a reaction aliquot indicated complete reduction and 70% para isomer in the regioisomeric mixture. The mixture was filtered through Celite®, and the filtrate was concentrated to an oil (41 g, 0.160 mol) that was subsequently diluted with THF (125 mL). This THF solution was added to a solution of ptoluenesulfonic acid monohydrate (37 g, 0.195 mol) in a 1:1 (v/v) THF/diethyl ether solution. Diethyl ether was added to this clear solution until the onset of cloudiness. After about 10 minutes, solids precipitated as a dense unstirrable mass. The mixture was diluted further with diethyl ether (300 mL) and THF (350 mL), and the resulting suspension was filtered. The filter cake was washed with 2:5 (v/v) THF/diethyl ether (3×80 mL) and the cake was dried under reduced pressure to afford [(2R)-2-(4-aminophenyl)propyl][(methylethyl)sulfonyl]amine p-toluenesulfonate (41.7 g, 54 %) as a white powder.

### EXAMPLE 5

### Alternative Preparation of ((2R)-2-phenylpropyl)[(methylethyl)sulfonyl]amine

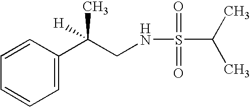

### Preparation of (2R)-2-phenylpropan-1-ol

An oven dried 500.0 mL three necked round bottom flask equipped with a mechanical stirrer, thermometer, addition funnel with a continuous nitrogen blanket is charged with 2.0 M solution of trimethylaluminum (65.6 mL, 131.2 mmol) and toluene (75.0 mL). Reaction solution was then chilled to −60° C. with dry ice/acetone bath. To this solution was then added R-styrene oxide dissolved in 100.0 mL of toluene over a period of 50.0 minutes (reaction is quite exothermic and can be controlled by the rate of addition of substrate). After stirring at this temperature for 60.0 minutes, reaction was brought to room temperature and stirred for 4.0 hours. Reverse quenched reaction at room temperature into a slurry of THF (100.0 mL) and sodium sulfate decahydrate (46.0 g) very cautiously over a period of 90.0 minutes (quenching was quite exothermic with evolution of gas). Filtered the precipitate formed over hyflo, then concentrated filtrate to provide the intermediate title compound, (2R)-2-phenylpropan-1-ol, (11.03 g, 92.6%) as an oil; ¹H nmr (CDCl₃, 300 MHz) δ 1.28-1.29 (d, 3H, J=6.9 Hz), 1.5 (b, 1H), 2.9-3.0 (m, 1H), 3.69-3.70 (d, 2H, J=6.64Hz), 7.24-7.35 (aromatic); ¹³C nmr (CDCl₃, 300 MHz) δ 18.31, 43.15, 69.40, 127.38, 128.20, 129.26 144.39.

### Preparation of 2-((2R)-2-phenylpropyl)isoindoline-1,3-dione

An oven dried 250.0 mL three necked round bottom flask equipped with a mechanical stirrer, thermometer, addition funnel with a continuous nitrogen blanket is charged with (2R)-2-phenylpropan-1-ol (2.0 mL, 14.32 mmol), phthalimide (2.1 g, 14.32 mmol), triphenylphosphine (5.63 g, 21.48 mmol) and THF (70.0 mL). To this solution at room temperature was then added a solution of diethylazodicarboxylate (3.38 mL, 21.48 mmol) dissolved in THF (10.0 mL) over a period of 15-20 minutes (reaction exothermed slightly to 50° C. by the end of addition went from clear to reddish color). Stirred reaction to room temperature overnight). To the red solution was added water (50.0 mL) and the organic extracted with chloroform (140.0 mL). Dried the organic solution with anhydrous magnesium sulfate, filtered and concentrated under reduced pressure to an oil. To the oil was added heptane (150.0 mL) with stirring. Filtered of precipitates, then concentrated filtrate to an oil. Plug filtration of the oil over silica gel with 1:1 ethylacetate/hexane and concentrating product fractions afforded the interrmhediate title compound, 2-((2R)-2-phenylpropyl)isoindoline-1,3-dione, (4.27 g, 96%) as an oil which solidified on equilibrating to room temperature; ¹H nmr (CDCl₃, 300 MHz) δ 1.3 (d, 3H), 3.3-4.0 (m, 1H), 3.7-3.9 (m, 2H), 7.1-7.3 (aromat. m, 2H), 7.63-7.7 (aromat. m, 2H), 7.8-7.85 (aromat. m, 4H).

### Preparation of (2R)-2-phenylpropylamine

A 500 mL three necked round bottom flask equipped with a mechanical stirrer, thermometer and addition funnel is charged with 2-((2R)-2-phenylpropyl)isoindoline-1,3-dione (11.54 g, 43.49 mmol), toluene (200.0 mL) and anhydrous hydrazine (2.73 mL, 86.99 mmol). Reaction is then stirred at room temperature for 3.0 hours and then heated at 90° C.-95° C. for 2.0 hours. Cooled the slurry to room temperature, filtered precipitates, then concentrated filtrate to provide the intermediate title compound, (2R)-2-phenylpropylamine, (5.58 g, 94.9%) an oil; ¹H nmr (CDCl₃, 300 MHz) δ 1.21 (d, 3H), 1.40-1.60 (b, 2H), 2.68-2.80 (m, 1H), 2.81-2.87 (m, 2H) 7.20 (m, 2H), 7.32 (m, 2H).

### Preparation of Final Title Compound

To a solution of the (2R)-2-phenylpropylamine (1.2 g, 8.87 mmol) in hexane (16.0 mL) was added triethylamine (2.47 mL, 17.74 mmol) and dimethylaminopyridine (0.30 g, 2.47 mmol). Cooled reaction to 5° C., then added a solution of isopropylsulfonyl chloride (0.97 mL, 8.69 mmol) dissolved in methylene chloride (6.0 mL) over a period of 15.0 minutes. Stirred for 45.0 minutes, then stirred at room temperature for 120.0 minutes. Quenched reaction with 1N HCl (20.0 mL) and extracted organic with methylene chloride (25.0 mL). Dried organic layer with anhydrous magnesium sulfate, filtered and concentrated filtrate to provide the final title compound, ((2R)-2-phenylpropyl)[(methylethyl)sulfonyl]amine, (1.93 g, 90.1%) an oil; ¹H nmr (CDCl₃, 300 MHz) δ 1.25 (d, 3H, J=6.9Hz), 1.29(d, 3H, J=6.9Hz), 1.30 (d, 3H, J=7.2Hz), 2.98 (m, 1H), 3.05 (m, 1H), 3.22 (m, 1H), 3.36 (m, 1H), 3.89 (b, 1H), 7.23 (m, 2H), 7.34 (m, 2H).

### EXAMPLE 6

### Preparation of {(2R)-2-[4-(4-{2-[(methylsulfonyl)amino]ethyl}phenyl)phenyl]propyl}[(methylethyl)sulfonyl]amine

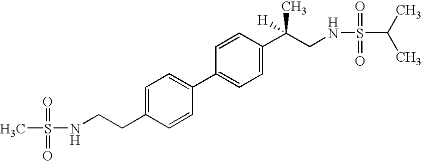

### Preparation of 2-Phenyl-1-propylamine HCl

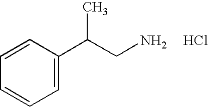

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitate solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder.

¹H NMR analysis of the free base: ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

### Preparation of (2R)-2-phenylpropylamine malate

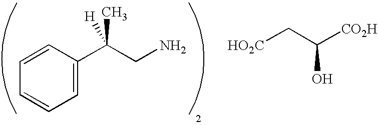

To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison with commercial 2-phenyl-1-propylamine) ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10 H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

### Preparation of ((2R)-2-phenylpropyl)[(methylethyl)sulfonyl]amine

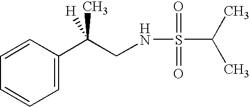

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J=6.9), 1.25 (d, 3H, J=6.9).

### Preparation of [(2R)-2-(4-iodophenyl)propyl][(methylethyl)sulfonyl]amine

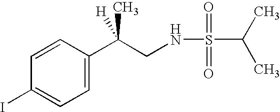

A stirred room temperature solution of ((2R)-2-phenylpropyl)[(methylethyl)sulfonyl]amine (37.1 g, 0.154 mol) in glacial acetic acid (185 mL) was treated with concentrated H₂SO₄ (16.0 g, 0.163 mol), added dropwise in a slow stream, followed by a H₂O rinse (37 mL). To this solution (˜30° C.) was added H₅IO₆ (8.29 g, 0.0369 mol), followed by iodine (17.9 g, 0.0707 mol). The resulting reaction mixture was heated and allowed to stir for 3 h at 60° C. After HPLC analysis verified the consumption of starting material, the reaction mixture was cooled to 300 C and a 10% aqueous solution of NaHSO₃ (220 mL) was added dropwise while maintaining the temperature between 25° C. and 30° C. The mixture crystallized to a solid mass upon cooling to 0-5° C.

The solids were suction filtered and rinsed with H₂O to afford 61.7 g of crude solids that were redissolved into warm MTBE (500 mL). This solution was extracted with H₂O (2×200 mL) and saturated NaHCO₃ (1×200 mL) and the organic phase was dried (MgSO₄), filtered, and concentrated under reduced pressure to 200 mL. Heptane (100 mL) was added dropwise to the product solution with slow stirring until crystallizatiori commenced. An additional 100 mL of heptane was added and the resulting suspension was allowed to stir slowly overnight at room temperature. The mixture was then cooled (0° C.), filtered, and the collected solids were rinsed with heptane. The solids were then air-dried to afford the intermediate title compound, [(2R)-2-(4-iodophenyl)propyl][(methylethyl)sulfonyl]amine (33.7 g, 59.8%) as a white powder. Chiral Chromatography of this lot indicated 100% e.e.

¹H NMR (CDCl₃, 300 MHz) δ 7.66 (d, 2H, J=8.1), 6.98 (d, 2H, J=8.4), 3.86 (br t, 1H, J=5.1), 3.33 (m, 1H), 3.18 (m, 1H), 3.06 (m, 1H), 2.92 (m, 1H), 1.30 (d, 3H, J=6.6), 1.27 (d, 6H, J=6.6).

### Preparation of (methylsulfonyl)(2-phenylethyl)amine

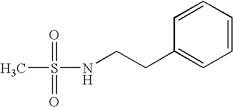

To a 10° C. solution of phenethylamine (12.1 g, 0.1 00 mol) and triethylamine (11.1 g, 0.110 mol) in CH₂Cl₂ (50 mL) was added methanesulfonyl chloride (12.6 g, 0.110 mol) dropwise over 10 min. The solution was stirred at room temperature for 1.5 h and was then washed with 1 N HCl (5×20 mL). The organic phase was directly concentrated to provide the intermediate title compound, (methylsulfonyl)(2-phenylethyl)amine, (21.2 g, 93.3%) as an oil. ¹H NMR (CDCl₃, 300 MHz) δ 7.32 (m, 2H), 7.23 (m, 3H), 4.30 (br s, 1H), 3.40 (t, 2H, J=3.9), 2.88 (t, 2H, J=4.2), 2.81 (s, 3H).

### Preparation of [2-(4-iodophenyl)ethyl](methylsulfonyl)amine

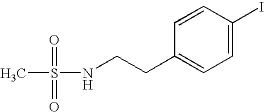

To a stirring room temperature solution of (methylsulfonyl)(2-phenylethyl)amine (205 g, 1.03 moles), water (200 mL), 95% sulfuric acid (111 g, 1.08 moles) in acetic acid (1 L), was added iodine (111 g, 0.438 mol) and periodic acid (H₅IO₆, 45.6 g, 0.206 mol). The reaction mixture was warmed to 70-75° C. for 3 h. The heat was removed and the dark violet reaction mixture was 2 0 allowed to proceed overnight at room temperature. Potassium hydroxide pellets (85%, 143 g, 2.16 moles) were added to neutralized the sulfuric acid and then enough saturated aqueous sodium sulfite was added to decolorize the mixture to afford a white suspension. The suspension was cooled to 15° C. and filtered.

The filter cake was triturated thoroughly with water and was then dissolved in CH₂Cl₂ (1 L) and extracted with additional water (2×200 mL). The organic phase was concentrated under reduced pressure to provide the intermediate title compound, [2-(4-iodophenyl)ethyl](methylsulfonyl)amine, (201 g, 60.2%) as a white powder.

¹H NMR (CDCl₃, 300 MHz) δ 7.64 (d, 2H, J=4.8), 6.97 (d, 2H, J=5.1), 4.37 (br t, 1H, J=4), 3.36 (app. q, 2H, J=3.9), 2.85 (s, 3H), 2.82 (t, 2H, J=3.9).

### Preparation of (tert-butoxy)-N-[2-(4-iodoghenyl)ethyl]-N-(methylsulfonyl)carboxamide

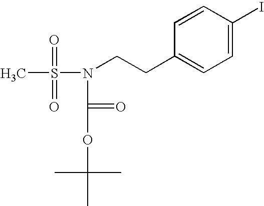

A room temperature solution of [2-(4-iodophenyl)ethyl](methylsulfonyl)amine (201 g, 0.618 mol), 4-dimethylaminopyridine (3.8 g, 0.031 mol) and di-teit-butyl dicarbonate (162 g, 0.744 mol) in CH₂Cl₂ (1 L) was allowed to stir overnight. The reaction mixture was washed with water (2×400 mL) and the organic phase was concentrated to about 600 mL and hexanes (400 mL) was added. This combined solution was washed again with water (400 mL) and was concentrated to a solid that was suspended in hexanes (600 mL) and filtered. The collected solids were dried under reduced pressure to afford the intermediate title compound, (tert-butoxy)-N-[2-(4-iodophenyl)ethyl]-N-(methylsulfonyl)carboxamide (241.5 g, 91.5%) as a white solid.

¹H NMR (CDCl₃, 300 MHz) δ 7.63 (d, 2H, J=7.8), 6.98 (d, 2H, J=7.8), 3.88 (t, 2H, J=6.9), 3.10 (s, 3H), 2.88 (t, 2H, J=6.9), 1.51 (s, 9H).

### Preparation of (tert-butoxy)-N-(methylsulfonyl)-N-{2-[4-(4,4,5,5-tetramethyl(1,3,2-dioxaborolan-2-yl))phenyl]ethyl}carboxamide

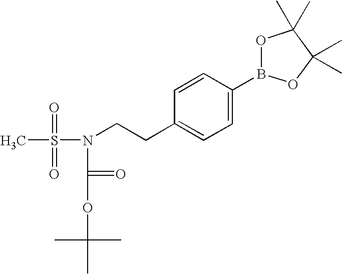

To a degassed solution of (tert-butoxy)-N-[2-(4-iodophenyl)ethyl)-N-(methylsulfonyl)carboxamide (128 g, 0.300 mol), triethylamine (91.1 g, 0.900 mol), and 1,1′-bis(diphenylphosphino) ferrocenedichloropalladium (II)-CH₂Cl₂ complex (2.9 g, 0.0035 mol) in acetonitrile (600 mL) was added pinacolborane (50 g, 0.391 mol) dropwise. The mixture was stirred at 70-74° C. for 8 h and then was cooled to room temperature. The reaction mixture was concentrated to a fluid oil that was partitioned between MTBE (500 mL) and water (500 mL). The organic phase was separated and washed with water (2×200 mL) and concentrated to a residue that was partially dissolved with heptane (1 L). The heptane soluble fraction was filtered through Celite® 521 and concentrated to an oil (95 g). The residue was dissolved in acetone (600 mL) and heptane (600 mL) and filtered through Celite® 521. The combined filtrates were concentrated to 95 g of a mixture of a 3:1 molar ratio (1H NMR, 81.0% by weight) of intermediate title compound, (tert-butoxy)-N-(methylsulfonyl)-N-{2-[4-(4,4,5,5-tetramethyl(1,3,2-dioxaborolan-2-yl))phenyl]ethyl}carboxamide, (60.3% potency corrected yield) and protio derivative.

¹H NMR (CDCl₃, 300 MHz) δ 7.75 (d, 2H, J=7.8), 7.23 (d, 2H, J=8.1), 3.87 (t, 2H, J=8.1), 2.99 (s, 3H), 2.90 (t, 2H, J=7.5), 1.53 (s, 9H), 1.33 (s, 6H), 1.27 (s, 6H).

### Preparation of (methylsulfonyl){2-[4-(4,4,5.5-tetramethyl(1,3,2-dioxaborolan-2-yl))phenyl]ethyl}amine

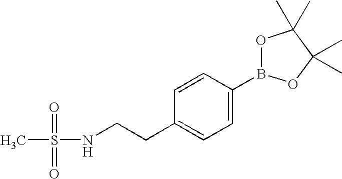

To a 2 L flask charged with a stirring solution of (tert-butoxy)-N-(methylsulfonyl)-N-{2-[4-(4,4,5,5-tetramethyl(1,3,2-dioxaborolan-2-yl))phenyl]ethyl}carboxamide (98.7 g, 0.232 mol) in CH₂Cl₂ (500 mL) was added trifluoroacetic acid (82 mL, 121.4 g, 1.06 moles) dropwise from an addition funnel. No exotherm was observed and the reaction solution was allowed to stir at room temperature for 18 h.

HPLC analysis indicated 98% completion so the cooled (5° C.) reaction mixture was neutralized by the slow addition of 5N NaOH (175 mL). The pH of the aqueous phase was 10.5. The phases were separated and the aqueous phase was extracted with CH₂Cl₂ (50 mL). The combined CH₂Cl₂ phases were washed with brine (2×100 mL) and water (1×100 mL). The CH₂Cl₂ phase was diluted with heptane (300 mL) and was concentrated under reduced pressure to afford a suspension that was isolated by filtration. The collected solids were washed with pentane (2×100 mL) and dried under vacuum to provide the intermediate title compound, (methylsulfonyl){2-[4-(4,4,5,5-tetramethyl(1,3,2-dioxaborolan-2-yl))phenyl]ethyl}amine, (69.0 g, 91.4%) as a white powder.

¹H NMR (CDC13, 300 MHz) δ 7.77 (d, 2H, J=8.1), 7.22 (d, 2H, J=7.8), 4.26 (br t, 1H, J=6), 3.40 (q, 2H, J=6.9), 2.89 (t, 2H, J=6.6), 2.82 (s, 3H), 1.34 (s, 12H).

### Preparation of 4-{2-[(methylsulfonyl)amino]ethyl}benzene boronic acid

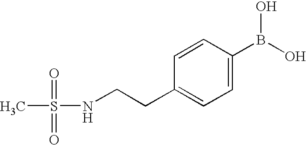

(Methylsulfonyl){2-[4-(4,4,5,5-tetramethyl(1,3,2-dioxaborolan-2-yl))phenyl]ethyl}amine (68.0 g, 0.209 mol) was placed into a 2L flask and combined with acetone (600 mL), 1N ammonium acetate (600 mL), and NaIO₄ (168.1 g, 0.786 mol). This mixture was stirred at room temperature overnight. The reaction mixture was filtered to remove insoluble matter to afford filtrate A. The collected solids were washed with acetone (2×100 mL) and this filtrate was combined with filtrate A. The combined filtrates were concentrated under reduced pressure to 600 mL to afford a precipitate that was recovered by filtration. The collected solids were air-dried to give 110 g of crude material. This crude material was suspended in water (100 mL) and 5N NaOH was added until the pH was 12.5. The resulting suspension was filtered and the filtrate was treated with decolorizing carbon (Darco 6-60). The mixture was filtered and the filtrate was diluted with 10N H₂SO₄ until the pH was 5.0 to precipitate the intermediate title compound. This precipitate was collected by filtration and dried under reduced pressure to provide the intermediate title compound, 4-{2-[(methylsulfonyl)amino]ethyl}benzene boronic acid, (41.9 g, 82.5%) as a white powder.

¹H NMR (acetone-d₆, 300 MHz) δ 7.82 (d, 2H, J=8.4), 7.27 (d, 2H, J=7.8), 7.11 (s, 2H), 6.03 (m, 1H), 3.36 (m, 2H), 2.91 (m, 2H), 2.84 (s, 3H).

### Preparation of Final Title Compound

An aqueous solution of potassium formate was prepared in the following manner. To 15 mL of water was added KOH (85% flakes, 6.73 g, 0.102 mol), then 98% formic acid (4.70 g, 0.102 mol). Alternatively, one may use commercially available potassium formate. To this solution was then added K₂CO₃ (2.76 g, 0.0210 mol), 4-{2-[(methylsulfonyl)amino]ethyl}benzene boronic acid (4.62 g, 0.190 mol), 1-propanol (100 mL), and [(2R)-2-(4-iodophenyl)propyl][(methylethyl)sulfonyl]amine (7.35 g, 0.200 mol). This mixture was deoxygenated via three vacuum/N₂-refill cycles. Palladium black (0.0215 g, 0.0002 mol) was added and the mixture was again deoxygenated via three vacuum/N₂-refill cycles. The reaction flask was heated in a preheated oil bath at 88° C. and the mixture was stirred overnight.

HPLC analysis showed complete consumption of 4-{2-[(methylsulfonyl)amino]ethyl}benzene boronic acid, and the mixture was diluted with ethyl acetate and filtered through Celite® to remove palladium. The mixture was concentrated under reduced pressure and the resulting residue was partitioned between ethyl acetate and water. The organic phase was concentrated and the solid residue was collected and recrystallized from 1:1 acetone/water to afford the final title compound, {(2R)-2-[4-(4-{2-[(methylsulfonyl)amino]ethyl}phenyl)phenyl]propyl}[(methylethyl)sulfonyl]amine, (6.2 g, 75%) as a white crystalline powder.

¹H NMR (CDCl₃, 300 MHz) δ 7.54 (dd, 4H, J=1.8, 8.1), 7.29 (dd, 4H, J=1.8, 8.1), 4.27 (t, 1H, J=6.6), 3.91 (m, 1H), 3.43 (q, 2H, J=6.6), 3.37 (dd, 1H, J=5.7, 7.5), 3.26 (m, 1H), 3.07 (m, 2H), 2.93 (t, 2H, J=6.6), 2.87 (s, 3H), 1.34 (d, 3H, J=7.2), 1.31 (d, 3H, J=6.9), 1.27 (d, 3H, J=6.6).

### Additional Preparation of {(2R)-2-[4-(4-{2-[(methylsulfonyl)amino]ethyl}phenyl)-phenyl]propyl}(methylethyl)sulfonyl]amine

Within a single-neck, 3 L round bottom flask equipped with a magnetic stir bar was placed potassium formate (112.8 g, 1.34 moles, 5.1 eq) and water (200 mL) to provide a pH 8 solution. Potassium carbonate (72.7 g, 0.526 mol, 2.0 eq), and 4-{2-[(methylsulfonyl)amino]ethyl}benzene boronic acid (60.8 g, 0.250 mol. 0.95 eq) was added to form a stirring suspension as 1-propanol (720 mL) was added. [(2R)-2-(4-iodophenyl)propyl][(methylethyl)sulfonyl]amine (96.6 g, 0.263 mol, 1.0 eq) was added followed by additional 1-propanol (600 mL). The resulting mixture was stirred for 3 minutes while the reaction flask was fitted with a heating mantle and a glycol-cooled reflux condenser. Vacuum (10-20 torr) was slowly applied to the system over 10 minutes. Stirring had stopped due to the additional precipitation of the cooled system; nevertheless, after 30 minutes, the system was returned to atmospheric pressure with nitrogen. With gentle heating, the flask was evacuated and refilled with nitrogen two additional times. Stirring was stopped and palladium black (0.28 g, 0.0026 mol, 0.01 eq) was quickly added to the flask. Stirring was resumed and the system was again evacuated and returned to atmospheric pressure with nitrogen over a 2 minute cycle. This evacuation/nitrogen purge was repeated two more times over a 15 second cycle and the mixture was heated to reflux.

After 16 hours, an aliquot was removed and analyzed by HPLC (275 nm detection). Analysis showed 0.07% of achiral dimer, (methylsulfonyl){2-[4-(4-{2-[(methylsulfonyl)amino]ethyl}phenyl)phenyl]ethyl}amine, relative to the desired product, {(2R)-2-[4-(4-{2-[(methylsulfonyl)amino]ethyl}phenyl)phenyl]propyl}[(methylethyl)sulfonyl]amine. The reaction mixture was cooled to 50° C. and ethyl acetate (500 mL) was added. The reaction mixture was then cooled to room temperature and the product, {(2R)-2-[4-(4-{2-[(methylsulfonyl)amino]ethyl}phenyl)phenyl]propyl}[(methylethyl)sulfonyl]amine, began to precipitate. Additional ethyl acetate (1 L) was introduced to redissolve the product and the upper organic phase was decanted and filtered through Celite® to remove palladium metal. The filter cake was rinsed with 1-propanol. The homogeneous filtrate was concentrated under reduced pressure to remove n-propanol and after removal of 1.5 L of distillate, the product suspension was filtered. The combined filter cakes were dried to afford 109.8 g of crude final title compound.

Recrystallization: The crude final title compound (109.8 g) was dissolved in acetone (490 mL). This solution was filtered though a glass filter to retain a minor amount of dark insoluble material. To the slowly stirred filtrate was added water (300 mL) over 15 min. The resulting suspension was stirred for 15 minutes and additional water (20 mL) was introduced over 10 minutes. The suspension was subsequently stirred for 30 minutes at room temperature and was filtered. The cake was washed with 1:1 acetone/water (600 mL) and was dried at 35° C. overnight. This process afforded 80.3 g (81.1%) of {(2R)-2-[4-(4-{2-[(methylsulfonyl)amino]ethyl}phenyl)phenyl]propyl}[(methylethyl)sulfonyl]amine as a white crystalline powder with a mean particle size of about 29 to about 34 microns. HPLC analysis indicated 0.01% achiral dimer, (methylsulfonyl){2-[4-(4-{2-[(methylsulfonyl)amino]ethyl}phenyl)phenyl]ethyl}amine, and 0.02% chiral dimer, ((2R)-2-{4-[4-((1R)-1-methyl-2-{[(methylethyl)sulfonyl]amino}ethyl)phenyl]phenyl}propyl)[(methylethyl)sulfony]amine.

### EXAMPLE 7

### Alternative Preparation of {(2R)-2-[4-(4-{2-[(methylsulfonyl)amino]ethyl}phenyl)phenyl]propyl}[(methylethyl)sulfonyl]amine

### Preparation of 4-{2-[(tert-butoxy)-N-(methylsulfonyl)carbonylamino]ethyl}benzene boronic acid

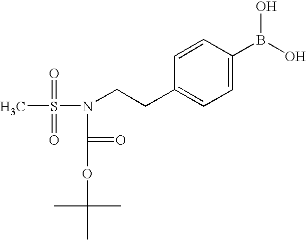

To a room temperature solution of (tert-butoxy)-N-(methylsulfonyl)-N-{2-[4-(4,4,5,5-tetramethyl(1,3,2-dioxaborolan-2-yl))phenyl]ethyl}carboxamide (81.0% potent, 95 g, 0.18 mol, prepared in example 1) in acetone (2 L) was added 1N ammonium acetate (1 L) and sodium periodate (145 g, 0.678 mol) with stirring. The reaction was allowed to proceed overnight. The reaction mixture was concentrated to remove the acetone, and the aqueous phase was decanted away from the oily product. The aqueous phase was extracted with CH₂Cl₂ (100 mL) and MTBE (2×100 mL). The combined oily product and organic phases were adjusted to pH 12.5 with the addition of 1 N NaOH. The phases were separated, and the organic phase was extracted with 1 N NaOH (100 mL) and water (2×100 mL). HPLC analysis (60% CH₃CN/40% H₂O, 2 mL/min, Zorbax C-18, 205 nm) of the organic phase indicated that the product had been removed from this phase. The aqueous phases (containing product) were finally combined and washed with CH₂Cl₂ (100 mL) and MTBE (2×100 mL). The aqueous phase was added to CH₂Cl₂ (450 mL) and 1 N H₂SO₄ was added until the aqueous phase was at pH 3.05. The phases were separated and the aqueous phase was extracted with CH₂Cl₂ (100 mL). The combined organic extracts (containing product) were concentrated to an oil (58.5 g) that crystallized overnight. The resulting solid mass was triturated with 10% MTBE in heptane (100 mL) to afford, after filtration and drying under reduced pressure, the intermediate title compound, 4-{2-[(tert-butoxy)-N-(methylsulfonyl)carbonylamino]ethyl}benzene boronic acid, (47.7 g, 77.2%) as a white powder.

¹H NMR (d₆-DMSO, 300 MHz) δ 7.83 (d, 2H, J=4.8), 7.24 (d, 2H, J=5.1), 7.12 (s, 2H), 3.90 (t, 2H, J=3.9), 3.12 (s, 3H), 2.95 (t, 2H, J=4.5), 1.52 (s, 9H).

### Preparation of Final Title Compound

Run 1. Within a 3-neck, 1000 mL round-bottom flask was placed [(2R)-2-(4-iodophenyl)propyl][(methylethyl)sulfonyl]amine (15.0 g, 0.0408 mol, prepared in example 1), 4-{2-[(tert-butoxy)-N-(methylsulfonyl)carbonylamino]ethyl}benzene boronic acid (19.1 g, 0.0557 mol), K₂CO₃ (6.8 g, 0.0490 mol) and 1-propanol (300 mL). To this mixture was then added water (42 mL) and finally Pd(OAc)₂ (18 mg, 8.17×10⁻⁵ mol, 0.2 mol %). The resulting clear, pale amber solution was heated to reflux (87° C.) to become a dark amber, then a clear olive solution with stirring black particulates (Pd°). The reaction was allowed to stir for 20 h and was allowed to cool to room temperature. TLC analysis (1:9 EtOAc/CH₂Cl₂) of the resulting off-white suspension indicated desired product (R_{f} 032), complete consumption of [(2R)-2-(4-iodophenyl)propyl][(methylethyl)sulfonyl]amine (R_{f} 0.60) and only a trace of 4-{2-[(tert-butoxy)-N-(methylsulfonyl)carbonylamino]ethyl}benzene boronic acid (R_{f} 0.49). The suspension was diluted with EtOAc (300 mL) to give a clear, pale yellow solution that was filtered through Celite® (presaturated with EtOAc).

After washing the Celite® through with EtOAc, the filtrate was combined with that of an identical Run 2 which was conducted identically as described above. The combined filtrates from both runs were concentrated under reduced pressure to afford white solids that were diluted with EtOAc (1 L) and 10% K₂CO₃ (300 mL) to form a clear, amber biphasic solution that was agitated. The aqueous phase (light pink) was separated and the organic phase was washed with additional 10% K₂CO₃ (4×300 mL). The aqueous phase was back extracted with EtOAc (300 mL) and the combined organic phases (1500 mL) were dried (MgSO₄), filtered, and concentrated to a volume of about 620 mL within a 3 L round-bottom flask. The clear, pale yellow solution was stirred slowly while heating to 60° C. Heptane (400 mL) was added dropwise from a separatory funnel to the stirring EtOAc solution at 60° C. (17 volumes of EtOAc/11 volumes of heptane). The heptanes were added over a period of 1.5 h and the clear, pale yellow solution was allowed to cool slowly with slow stirring overnight. The resulting white crystalline solids were cooled to 0° C., filtered, and washed with a minimum of 1:1 EtOAc/heptanes to afford the final title compound, {(2R)-2-[4-(4-{2-[(methylsulfonyl)amino]ethyl}phenyl)phenyl]propyl}[(methylethyl)sulfonyl]amine, (27.1 g, 75.7%) as a white crystalline powder.

### EXAMPLE 8

### Alternative Preparation of ((2R)-2-phenylpropyl)[(methylethyl)sulfonyl]amine

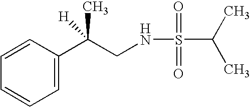

### Preparation of (2R)-2-phenylpropan-1-ol

An oven dried 500.0 mL three necked round bottom flask equipped with a mechanical stirrer, thermometer, addition funnel with a continuous nitrogen blanket is charged with 2.0 M solution of trimethylaluminum (65.6 mL, 131.2 mmol) and toluene (75.0 mL). Reaction solution was then chilled to −60° C. with dry ice/acetone bath. To this solution was then added R-styrene oxide dissolved in 100.0 mL of toluene over a period of 50.0 minutes (reaction is quite exothermic and can be controlled by the rate of addition of substrate). After stirring at this temperature for 60.0 minutes, reaction was brought to room temperature and stirred for 4.0 hours. Reverse quenched reaction at room temperature into a slurry of THF (100.0 mL) and sodium sulfate decahydrate (46.0 g) very cautiously over a period of 90.0 minutes (quenching was quite exothermic with evolution of gas). Filtered the precipitate formed over hyflo, then concentrated filtrate to provide the intermediate title compound, (2R)-2-phenylpropan-1-ol, (11.03 g, 92.6%) as an oil; ¹H nmr (CDCl₃) δ 1.28-1.29 (d, 3H, J=6.9Hz), 1.5 (b, 1H), 2.9-3.0 (m, 1H), 3.69-3.70 (d, 2H, J=6.64Hz), 7.24-7.35 (aromatic); ¹³C nmr (CDCl₃) δ 18.31, 43.15, 69.40, 127.38, 128.20, 129.26 144.39.

### Preparation of 2-((2R)-2-phenylpropylisoindoline-1,3-dione

An oven dried 250.0 mL three necked round bottom flask equipped with a mechanical stirrer, thermometer, addition funnel with a continuous nitrogen blanket is charged with (2R)-2-phenylpropan-1-ol (2.0 mL, 14.32 mmol), phthalimide (2.1 g, 14.32 mmol), triphenylphosphine (5.63 g, 21.48 mmol) and THF (70.0 mL). To this solution at room temperature was then added a solution of diethylazodicarboxylate (3.38 mL, 21.48 mmol) dissolved in THF (10.0 mL) over a period of 15-20 minutes (reaction exothermed slightly to 50° C. by the end of addition went from clear to reddish color). Stirred reaction to room temperature overnight). To the red solution was added water (50.0 mL) and the organic extracted with chloroform (140.0 mL). Dried the organic solution with anhydrous magnesium sulfate, filtered and concentrated under reduced pressure to an oil. To the oil was added heptane (150.0 mL) with stirring. Filtered of precipitates, then concentrated filtrate to an oil. Plug filtration of the oil over silica gel with 1:1 ethylacetate/hexane and concentrating product fractions afforded the intermediate title compound, 2-((2R)-2-phenylpropyl)isoindoline-1,3-dione, (4.27 g, 96%) as an oil which solidified on equilibrating to room temperature; ¹H nmr (CDCl₃) δ 1.3 (d, 3H), 3.3-4.0(m, 1H), 3.7-3.9 (m, 2H), 7.1-7.3 (aromat. m, 2H), 7.63-7.7 (aromat. m, 2H), 7.8-7.85 (aromat. m, 4H).

### Preparation of (2R)-2-phenylpropylamine

A 500 mL three necked round bottom flask equipped with a mechanical stirrer, thermometer and addition funnel is charged with 2-((2R)-2-phenylpropyl)isoindoline-1,3-dione (11.54 g, 43.49 mmol), toluene (200.0 mL) and anhydrous hydrazine (2.73 mL, 86.99 mmol). Reaction is then stirred at room temperature for 3.0 hours and then heated at 90° C.-95° C. for 2.0 hours. Cooled the slurry to room temperature, filtered precipitates, then concentrated filtrate to provide the intermediate title compound, (2R)-2-phenylpropylamine, (5.58 g, 94.9%) an oil; ¹H nmr (CDCl₃) δ 1.21 (d, 3H), 1.40-1.60 (b, 2H), 2.68-2.80 (m, 1H), 2.81-2.87 (m, 2H) 7.20 (m, 2H), 7.32 (m, 2H).

### Preparation of Final Title Compound

To a solution of the (2R)-2-phenylpropylamine (1.2 g, 8.87 mmol) in hexane (16.0 mL) was added triethylamine (2.47 mL, 17.74 mmol) and dimethylaminopyridine (0.30 g, 2.47 mmol). Cooled reaction to 5° C., then added a solution of isopropylsulfonyl chloride (0.97 mL, 8.69 mmol) dissolved in methylene chloride (6.0 mL) over a period of 15.0 minutes. Stirred for 45.0 minutes, then stirred at room temperature for 120.0 minutes. Quenched reaction with 1N HCl (20.0 mL) and extracted organic with methylene chloride (25.0 mL). Dried organic layer with anhydrous magnesium sulfate, filtered and concentrated filtrate to provide the final title compound, ((2R)-2-phenylpropyl)[(methylethyl)sulfonyl]amine, (1.93 g, 90.1%) an oil; ¹H nmr (CDCl₃) δ 1.25 (d, 3H, J=6.9 Hz), 1.29 (d, 3H, J=6.9 Hz), 1.30 (d, 3H, J=7.2 Hz), 2.98 (m, 1H), 3.05 (m, 1H), 3.22 (m, 1H), 3.36 (m, 1H), 3.89 (b, 1H), 7.23 (m, 2H), 7.34 (m, 2H).

### EXAMPLE 9

### Preparation of {(2S)-2-[4-(4-{2-[(methylsulfonyl)amino]ethyl}phenyl)phenyl]propyl}[(methylethyl)sulfonyl]amine

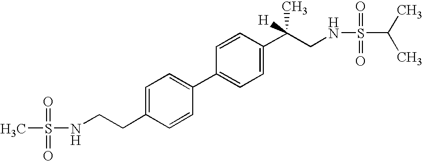

The title compound can be prepared in a manner analogous to the procedure set forth above in example 6 from (2S)-2-phenylpropylamine.

### EXAMPLE 10

### Preparation of 2-[4-(4-{2-[(methylsulfonyl)amino]ethyl}phenyl)phenyl]propyl}[(methylethyl)sulfonyl]amine

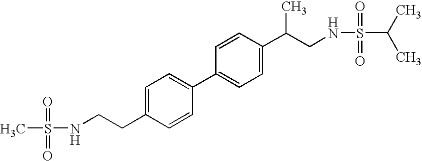

The title compound can be prepare, for example, following the procedure set forth in International Patent Application Publication WO 98/33496 published Aug. 6, 1998 at Example 51.

The ability of compounds A, B, C, D, E, and F, or the corresponding active metabolites to potentiate glutamate receptor-mediated response may be determined using fluorescent calcium indicator dyes (Molecular Probes, Eugene, Oregon, Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 mL of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl₂, 2 CaCl₂, 10 glucose, pH=7.4 with NaOH, 295 mOsm kg-1. The intracellular recording solution contains (in mM): 140 CsCl, 1 MgCl₂, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg-1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate.

In order to determine the potency of test compounds, the concentration of the test compound, both in the bathing solution and co-applied with glutamate, is increased in half log units until the maximum effect was seen. Data collected in this manner are fit to the Hill equation, yielding an EC₅₀ value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

According to another aspect, the present invention provides a pharmaceutical composition, which comprises a compound A, B, C, D, E, or F, or the corresponding active metabolites, or a pharmaceutically acceptable salt thereof and a pharmaceutically acceptable diluent or carrier.

The pharmaceutical compositions are prepared by known procedures using well-known and readily available ingredients. In making the compositions of the present invention, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, elixirs, suspensions, emulsions, solutions, syrups, aerosols, ointments containing, for example, up to 10% by weight of active compound, soft and hard gelatin capsules, suppositories, sterile injectable solutions, and sterile packaged powders.

Some examples of suitable carriers, excipients, and diluents include lactose, dextrose, sucrose, sorbitol, mannitol, starches, gum, acacia, calcium phosphate, alginates, tragcanth, gelatin, calcium silicate, micro-crystalline cellulose, polyvinylpyrrolidone, cellulose, water syrup, methyl cellulose, methyl and propyl hydroxybenzoates, talc, magnesium stearate, and mineral oil. The formulations can additionally include lubricating agents, wetting agents, emulsifying and suspending agents, preserving agents, sweetening agents, or flavoring agents. Compositions of the invention may be formulated so as to provide quick, sustained, or delayed release of the active ingredient after administration to the patient by employing procedures well known in the art.

The compositions are preferably formulated in a unit dosage form, each dosage containing from about 0.5 mg to about 50 mg, more preferably about 1 mg to about 20 mg of the compound A, B, C, D, E, or F, or the corresponding active metabolite, or a pharmaceutically acceptable salt thereof, most preferably about 1 mg to about 10 mg, and most especially preferably about 1 mg to about 5 mg. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical carrier, diluent, or excipient.

A typical daily dose will contain from about 0.5 mg to about 50 mg of the compound A, B, C, D, E, or F, or the corresponding active metabolite, or a pharmaceutically acceptable salt thereof. Preferably, daily doses will be about 1 mg to about 20 mg, most preferably from about 1 mg to about 10 mg, and most especially preferably about 1 mg to about 5 mg. It is understood that the daily dose may consist of administration to the patient of one, two, three, or four unit dosages per day.

As used herein the term “patient” refers to a mammal, such as a mouse, guinea pig, rat, dog or human. It is understood that the preferred patient is a human.

As used herein, the terms “treating” or “to treat” each mean to alleviate symptoms, eliminate the causation either on a temporary or permanent basis, or to prevent or slow the appearance of symptoms of the named disorder. As such, the methods of this invention encompass both therapeutic and prophylactic administration.

As used herein, the term “effective amount” refers to the amount of compound A, B, C, D, E, or F, or an active metabolite, or a pharmaceutically acceptable salt thereof, which is effective, upon single or multiple dose administration to a patient, in treating the patient suffering from the named disorder.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds A, B, C, D, E, or F, or the corresponding metabolite, or the pharmaceutically acceptable salt thereof, can be administered by a variety of routes including oral, rectal, transdermal, subcutaneous, intravenous, intramuscular, bucal or intranasal routes. Alternatively, the compounds A, B, C, D, E, or F, or the corresponding active metabolite, or the pharmaceutically acceptable salt thereof may be administered by continuous infusion.

The following compounds are preferred for use in the present invention:

A) 2-[4-(4-{2-[(methylsulfonyl)amino]ethyl}phenyl)phenyl]propyl}[(methylethyl)sulfonyl]amine;

B) {(2R)-2-[4-(4-{2-[(methylsulfonyl)amino]ethyl}phenyl)phenyl]propyl}[(methylethyl)sulfonyl]amine;

D) N-2-(4-N-(3,5-Difluorobenzamido)phenyl)propyl-2-propanesulfonamide; and

E) N-[4-((1R)-1-methyl-2-{[(methylethyl)sulfonyl]amino}ethyl)phenyl](3,5-difluorophenyl)carboxamide.

The following compounds are most preferred for use in the present invention:

B) {(2R)-2-[4-(4-{2-[(methylsulfonyl)amino]ethyl}phenyl)phenyl]propyl}[(methylethyl)sulfonyl]amine; and

E) N-[4-((1R)-1-methyl-2-{[(methylethyl)sulfonyl]amino}ethyl)phenyl](3,5-difluorophenyl)carboxamide.

1. A method of treating Bipolar I Disorder, Single Manic Episode; Bipolar I Disorder, Most Recent Episode Hypomanic; Bipolar I Disorder, Most Recent Episode Manic; Bipolar I Disorder, Most Recent Episode Mixed; Bipolar I Disorder, Most Recent Episode Depressed; Bipolar I Disorder, Most Recent Episode Unspecified; Bipolar II Disorder; or Bipolar Disorder NOS, in a patient, comprising administering to said patient an effective amount of a compound selected from the group consisting of:
2-[4-(4-{2-[(methylsulfonyl)amino]ethyl}phenyl)phenyl]propyl}[(methylethyl)sulfonyl]amine;
{(2R)-2-[4-(4-{2-[(methylsulfonyl)amino]ethyl}phenyl)phenyl]propyl}[(methylethyl)sulfonyl]amine;
N-2-(4-N-(3,5-Difluorobenzamido)phenyl)propyl-2-propanesulfonamide; and
N-[4-((1R)-1-methyl-2-{[(methylethyl)sulfonyl]amino}ethyl)phenyl](3,5-difluorophenyl)carboxamide; and
the pharmaceutically acceptable salts thereof.

2. A method according to claim 1 wherein the compound is:
{(2R)-2-[4-(4-{2-[(methylsulfonyl)amino]ethyl}phenyl)phenyl]propyl}[(methylethyl)sulfonyl]amine.

3. A method according to claim 1 wherein the compound is:
N-[4-((1R)-1-methyl-2-{[(methylethyl)sulfonyl]amino}ethyl)phenyl](3,5-difluorophenyl)carboxamide.

4. A method of treating multiple sclerosis, in a patient, comprising administering to said patient an effective amount of a compound selected from the group consisting of:
2-[4-(4-{2-[(methylsulfonyl)amino]ethyl}phenyl)pheny]propyl}[(methylethyl)sulfonyl]amine;
{(2R)-2-[4-(4-{2-[(methylsulfonyl)amino]ethyl}phenyl)pheny]propyl}[(methylethyl)sulfonyl]amine;
N-2-(4-N-(3,5-Difluorobenzamido)phenyl)propyl-2-propanesulfonamide; and
N-[4-((1R)-1-methyl-2-{[(methylethyl)sulfonyl]amino}ethyl)phenyl](3,5-difluorophenyl)carboxamide; and
the pharmaceutically acceptable salts thereof.

5. A method according to claim 4 wherein the compound is:
{(2R)-2-[4-(4-{2-[(methylsulfonyl)amino]ethyl}phenyl)phenyl]propyl}[(methylethyl)sulfonyl}amine.

6. A method according to claim 4 wherein the compound is:
N-[4-((1R)-1-methyl-2-{[(methylethyl)sulfonyl]amino}ethyl)phenyl](3,5-difluorophenyl)carboxamide.

7 and 8 (cancelled).
